(19) United States
(12) Patent Application Publication
(10) Pub. No.: US 2023/0139025 A1
(43) Pub. Date: May 4, 2023

(54) ANTIOXIDANT FOR USE IN TREATMENT FOR RIBE

(71) Applicant: Institute of Hematology and Blood Diseases Hospital, CAMS & PUMC, Tiangin (CN)

(72) Inventors: Tao Cheng, Tianjin (CN); Linping Hu, Tianjin (CN); Xiuxiu Yin, Tianjin (CN); Yawen Zhang, Tianjin (CN); Aiming Pang, Tianjin (CN); Xiaowei Xie, Tianjin (CN); Shangda Yang, Tianjin (CN); Caiying Zhu, Tianjin (CN); Yapu Li, Tianjin (CN); Biao Zhang, Tianjin (CN); Yaojin Huang, Tianjin (CN); Yunhong Tian, Tianjin (CN); Mei Wang, Tianjin (CN); Wenbin Cao, Tianjin (CN); Shulian Chen, Tianjin (CN); Yawei Zheng, Tianjin (CN); Shihui Ma, Tianjin (CN); Fang Dong, Tianjin (CN); Sha Hao, Tianjin (CN); Sizhou Feng, Tianjin (CN); Yongxin Ru, Tianjin (CN); Hui Cheng, Tianjin (CN); Erlie Jiang, Tianjin (CN)

(73) Assignee: Institute of Hematology and Blood Diseases Hospital, CAMS & PUMC, Tianjin (CN)

(21) Appl. No.: 17/518,590

(22) Filed: Nov. 4, 2021

(51) Int. Cl. A61K 31/198; A61K 31/26; A61K 31/05

(52) U.S. Cl. CPC A61K 31/198; A61K 31/05; A61K 31/26

(57) ABSTRACT

An antioxidant for use in treatment for a radiation-induced bystander effect to a transplanted cell in a host is provided. The antioxidant is N-acetyl-L-cysteine, sulforaphane or resveratrol, or a combination thereof. The RIBE on transplanted human hematopoietic cells impaired the long-term hematopoietic reconstitution of human HSCs as well as the colony-forming ability of HPCs, and the RIBE-affected human hematopoietic cells showed enhanced DNA damage responses, cell cycle arrest and p53-dependent apoptosis, mainly due to oxidative stress. Taken together, these findings suggest that RIBE impairs human HSCs by oxidative DNA damage. The present disclosure provides definitive evidence for RIBE in transplanted human HSCs and further justifies the necessity for conducting clinical trials to assess the ability of multiple antioxidants to improve the efficacy of HSC transplantation for patients with hematological or non-hematological disorders.

### TECHNICAL FIELD

The present invention relates to the treatment for radiation-induced bystander effects in hematopoietic stem cell transplantation, and particularly relates to an antioxidant for use in treatment for a radiation-induced bystander effect to a transplanted cell in a host.

### BACKGROUND

Transplantation of hematopoietic stem cells (HSCs) is a critical therapy for various malignant and nonmalignant hematological disorders and immune dysfunction. The key to successful transplantation is that the transplanted HSCs home to the host’s bone marrow (BM) niche and differentiate into multilineage mature blood cells, thus providing the patient with a revitalized hematopoietic and immune system.

Total body irradiation (TBI) is widely used for myeloablative conditioning regimens to eliminate malignant or autoimmune cells in acute lymphoblastic leukemia (ALL) or acute myeloid leukemia patients before HSC transplantation. TBI has remained the first choice in many centers for ALL. However, nonirradiated engrafted donor cells are also subject to damage impacting their survival and function, known as radiation-induced bystander effects (RIBE), which are caused by harmful factors transmitted by irradiated cells. Moreover, we have previously demonstrated acute negative bystander effects of irradiated recipients on transplanted mouse HSCs. Nevertheless, RIBE on human HSCs has not been established.

### SUMMARY OF THE INVENTION

The present invention reveals an antioxidant for use in treatment for a radiation-induced bystander effect to a transplanted cell in a host.

In a specific embodiment, the antioxidant is N-acetyl-L-cysteine, sulforaphane or resveratrol, or a combination thereof.

In a specific embodiment, the RIBE is in vivo or in vitro.

In a specific embodiment, the transplanted cell is a hematopoietic stem cell.

In a specific embodiment, the radiation-induced bystander effect to the transplanted cell is DNA damage or apoptosis of the transplanted cell caused by radiation to the host, or a combination thereof.

In a specific embodiment, the radiation-induced bystander effect to the transplanted cell is the transplanted cell’s reducing engraftment in the host.

In a specific embodiment, the radiation-induced bystander effect to the transplanted cell is a reduced hematopoietic stem cell enriched engraftment or a reduced hematopoietic progenitor cell enriched engraftment, or a combination thereof, in BM or SP, caused by radiation to the host.

In a specific embodiment, the antioxidant is sulforaphane or resveratrol, or a combination thereof.

In a specific embodiment, the radiation-induced bystander effect to the transplanted cell is the transplanted cell’s reduced erythroid differentiation potential.

In a specific embodiment, the antioxidant is resveratrol.

In a specific embodiment, the radiation-induced bystander effect to the transplanted cell is a reduced long-term repopulation potential or a reduced clonogenic potential in secondary transplantation.

### DETAILED DESCRIPTION OF THE FIGURES

FIG. 1 is an experimental diagram of in vivo RIBE model.

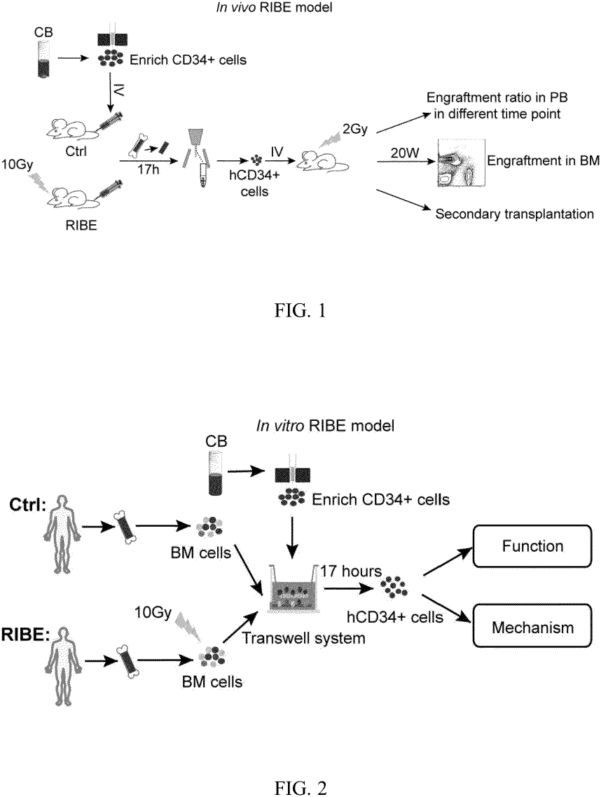

FIG. 2 is an experimental diagram of in vitro RIBE model.

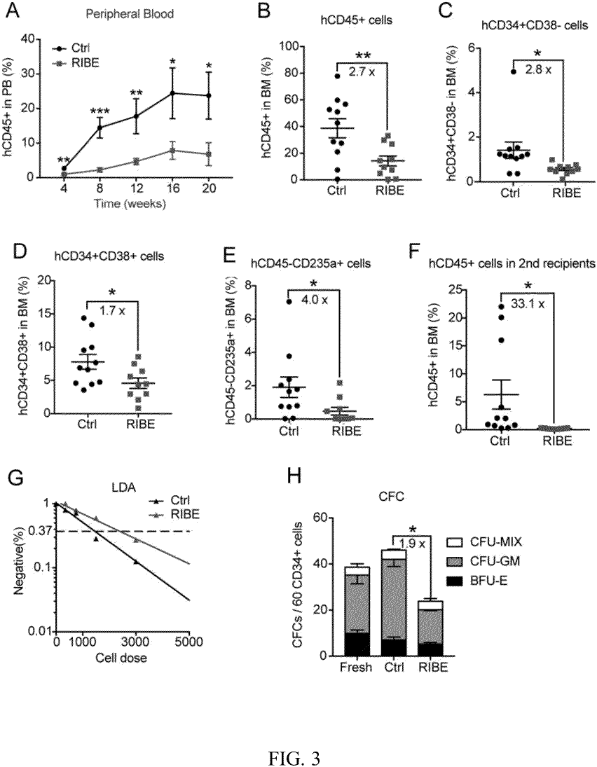

FIG. 3 shows that in vivo RIBE significantly reduces human long-term hematopoietic reconstitution and clonogenic ability, wherein the sub-figures shown are:

(A) Mean human cell engraftment level in peripheral blood (PB) at different time points after transplantation; (B) Percentage of human cell engraftment in the BM of recipients; (C) Frequency of human CD34⁺CD38⁻; (D) Frequency of human CD34⁺CD38⁺ cells in the BM of recipients; (E) frequency of human CD45⁻CD235a⁺ cells in the BM of recipients; (F-G) Percentage of human cell engraftment (F) and lineage differentiation potential (G) of the secondary mice (RIBE vs Ctrl: 0.19 ± 0.028% vs 6.29 ± 2.586%, p = 0.041; n = 9 to 11 per group); and (H) Number of hematopoietic colonies formed by hCD34⁺ cells in each group (n = 5 per group). (3 independent experiments; ^{∗}p < 0.05; ^{∗∗}p = 0.01 to 0.001; ^{∗∗∗}p < 0.001.)

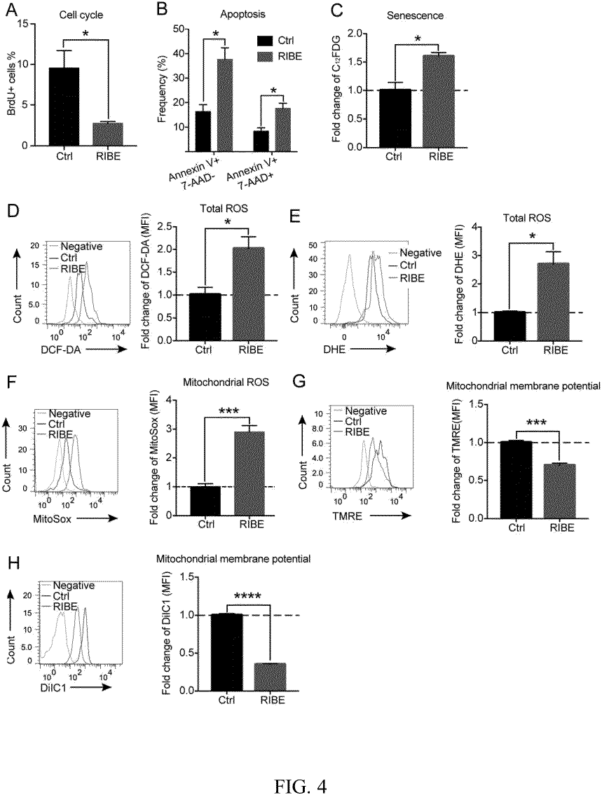

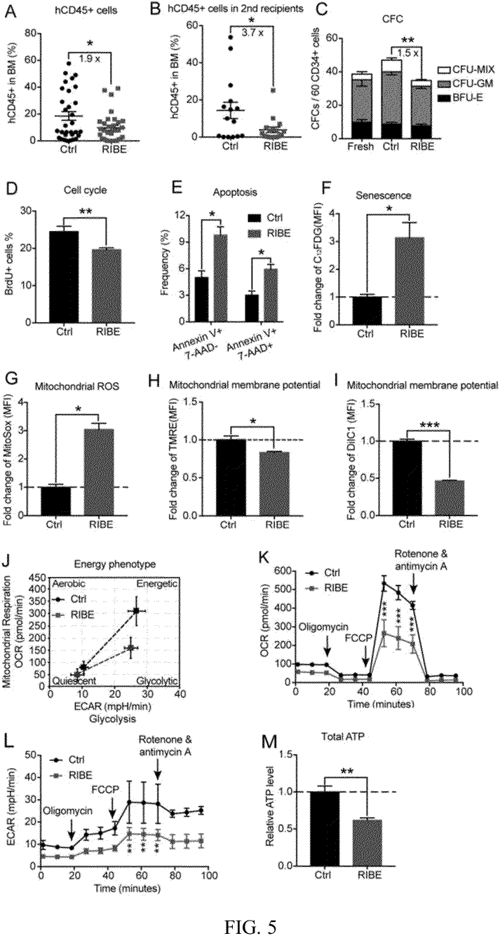

FIG. 4 shows that RIBE affects HSPCs′ cell cycle entry and increases their apoptosis and senescence in vivo, wherein the sub-figures shown are:

(A) The proportion of BrdU⁺ cells of human CD34⁺ cells from irradiated and nonirradiated mice. BrdU and human CD34⁺ cells were injected simultaneously; (B) Frequency of early apoptotic cells (Annexin V⁺7AAD⁻) and late apoptotic cells (Annexin V⁺7AAD⁺) in the homed human CD34⁺ cells from irradiated and nonirradiated mice; (C) Homed human CD34⁺ cells from irradiated and nonirradiated mice were sorted and cultured in vitro for three days to detect SA-β-gal activity. 5-dodecanoylaminofluorescein di-β-D-galactopyranoside (C₁₂FDG) was used as substrate. Bars represent fold change C₁₂FDG MFI compared with that in Ctrl group; (D-E) Flow cytometric analysis of fold change of ROS levels by DCF-DA (D) and DHE (E) staining in homed human CD34⁺ cells; (F) Fold change of mitochondrial ROS levels in homed human CD34⁺ cells detected by MitoSOX staining; and (G, H) Fold change of mitochondrial membrane potential of homed human CD34⁺ cells from irradiated or nonirradiated NOG mice determined with tetramethylrhodamine methyl ester (TMRE) (G) and DilC1(5) (H) staining. (n = 4 mice per group, ^{∗}p < 0.05; ^{∗∗}p = 0.01 to 0.001; ^{∗∗∗}p < 0.001.)

FIG. 5 shows that in vitro RIBE leads to defects of human HSPCs, wherein the sub-figures shown are:

(A) The frequency of human cell engraftment in BM 20 weeks after human CD34⁺ cells transplanted into NOG mice (RIBE vs Ctrl: 9.69 ± 1.903% vs 18.50 ± 3.301%, p = 0.023; n = 31 to 32 per group, 3 independent experiments); (B) Mean human cell engraftment in the BM of the secondary mice (RIBE vs ctrl, 3.87 ± 1.494 vs 14.35 ± 4.430, p = 0.025; n = 15 to 17 per group); (C) Number of hematopoietic colonies formed by human CD34⁺ cells in each group (n = 5 per group); (D) Proportion of BrdU⁺ cells of the human CD34⁺ cells from each group. BrdU was incubated with human CD34⁺ cells for 4 hours (n = 5 per group); (E) Frequency of early apoptotic cells (Annexin V⁺7AAD⁻) and late apoptotic cells (Annexin V⁺7AAD⁺) of human CD34⁺ cells from each group (n = 5 per group); (F) Fold change of SA- β -gal activity in human CD34⁺ cells analyzed using C12FDG as a substrate from each group (n = 5 per group); (G) Flow cytometric analysis of fold change in mitochondrial ROS levels by MitoSOX staining in human hematopoietic cells (n = 5 per group); (H, I) Fold change of mitochondrial membrane potential of human CD34⁺ cells determined by flow cytometry with TMRE (H) and DilC1(5) (I) staining (n = 5 per group); (J-L) The energy phenotype of CD34⁺ cells in the in vitro RIBE model (J), the oxygen consumption rate (OCR) (K) and extracellular acidification rate (ECAR) (L)assayed by Seahorse assay (n = 5 per group); and (M) Relative ATP levels in human CD34⁺ cells of in vitro RIBE model (n = 5 per group) (^{∗}p < 0.05, ^{∗∗}p < 0.01, ^{∗∗∗}p < 0.001).

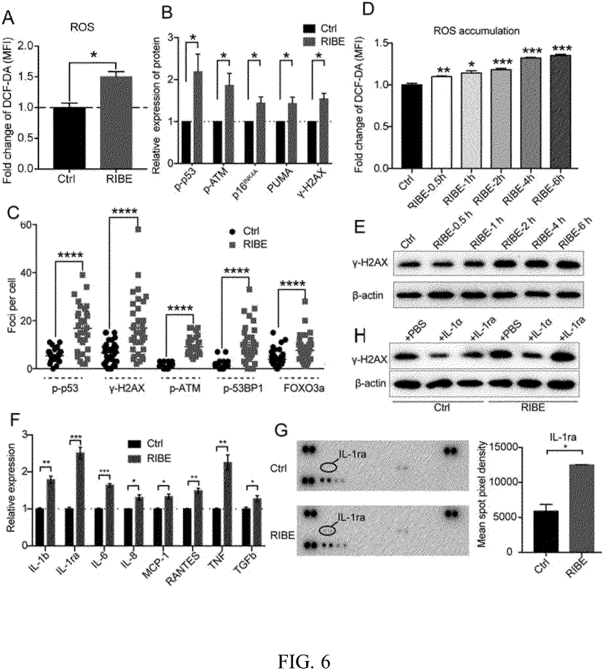

FIG. 6 shows that excessive ROS in bystander human hematopoietic cells results in DNA damage in HSPCs, wherein the sub-figures shown are:

(A) Flow cytometric analysis of fold change of ROS levels by DCF-DA staining in human CD34⁺ cells of in vitro RIBE model; (B) Western blot verified the expression of related signaling pathway at the protein levels. Each bar represents the mean ± S.D. for biological triplicate experiments; (C) Foci per cell from groups immunostained for p-p53, γ-H2AX, p-ATM, p-53BP1 and FOXO3a; (D) Flow cytometric analysis of fold change of ROS levels by DCF-DA staining of human CD34⁺ cells after different processing times; (E) γ-H2AX expression of CD34⁺ cells at the protein level after different processing times; (F) Relative RNA expression of cytokines of non-irradiated or irradiated human bone marrow cells (n = 3 per group); (G) Human cytokine array showed the relative expression of IL-1ra in non-irradiated or irradiated bone marrow supernatant, and the right panel is the quantification results; and (H) γ-H2AX expression of CD34⁺ cells at the protein level in different cytokine-treated groups (^{∗}p < 0.05, ^{∗∗}p < 0.01, ^{∗∗∗}p < 0.001).

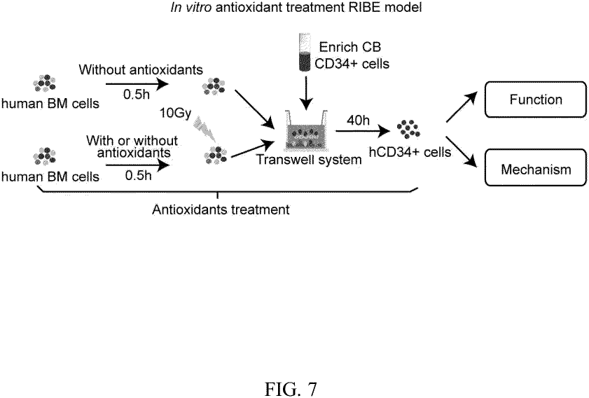

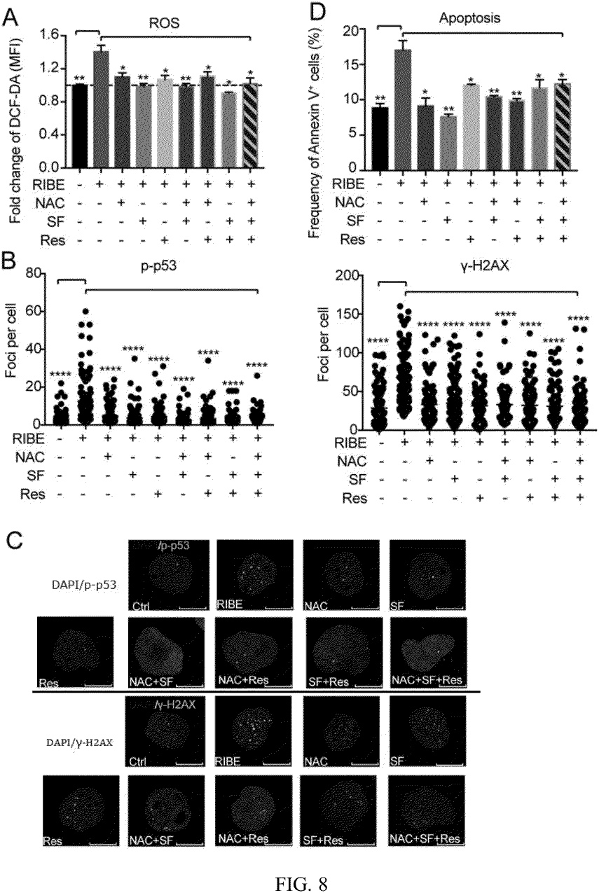

FIG. 7 is an experimental diagram of in vitro antioxidant treatment RIBE model. Human BM cells were treated with single antioxidant alone or in combination for 30 minutes before irradiation, and the antioxidants continued to exist in the coculture system for 40 hours.

FIG. 8 shows that inhibition of ROS elevation by antioxidant treatment loosens the functional deterioration of human HSPCs in an in vitro RIBE model, wherein the sub-figures shown are:

(A) Flow cytometric analysis of fold change of ROS levels by DCF-DA staining of human CD34⁺ cells from each group; (B-C) Human CD34⁺ cells from each group immunostained for p-p53 and γ-H2AX (p-p53 and γ-H2AX: green; DAPI: blue). Scatter plots represent foci per cell from each group (scale bar, 7 µm); and (D) Frequency of Annexin V⁺ cells of human CD34⁺ cells from each group.

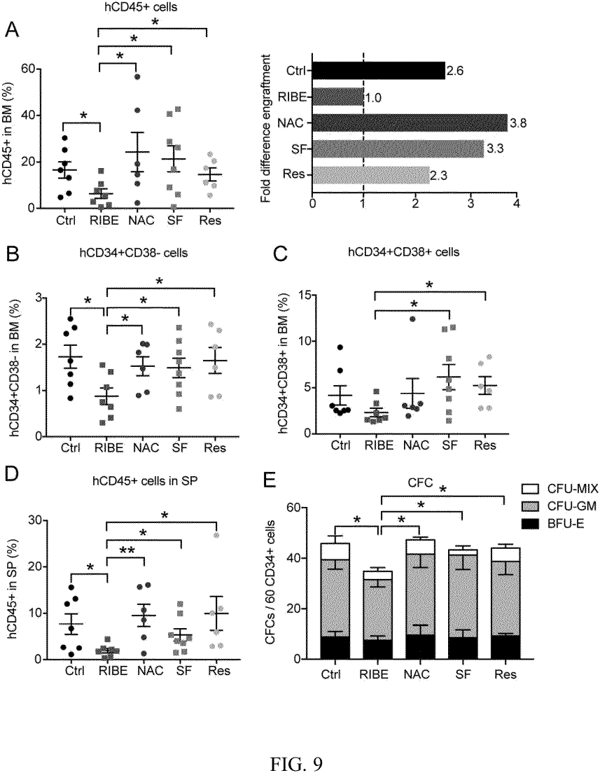

FIG. 9 shows that inhibition of ROS elevation protects the human HSPCs from functional deterioration in an in vivo RIBE model, wherein the sub-figures shown are:

(A) Collected human CD34⁺ cells from FIG. 8(A) were transplanted into NOG mice. Mean human cell engraftment (left) after human CD34⁺ cells collected from FIG. 8(A) transplanted into NOG mice and bars represent the fold difference of engraftment levels from each group (right, n = 6 to 8 per group). (B-C) Frequency of human CD34⁺CD38⁻ cells (B) and CD34⁺CD38⁺ cells (C) in the BM of recipient; (D) Mean engraftment level of human cells in the spleen of recipients; and (E) Number of hematopoietic clones formed by human CD34⁺ cells of each group (^{∗}p < 0.05, ^{∗∗}p <704 0.01, ^{∗∗∗}p < 0.001).

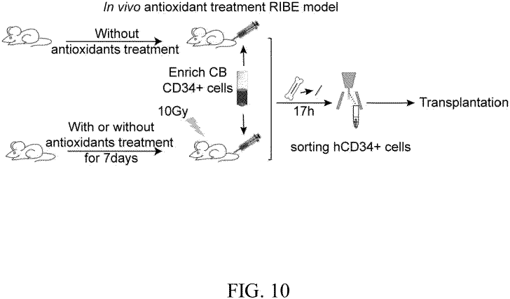

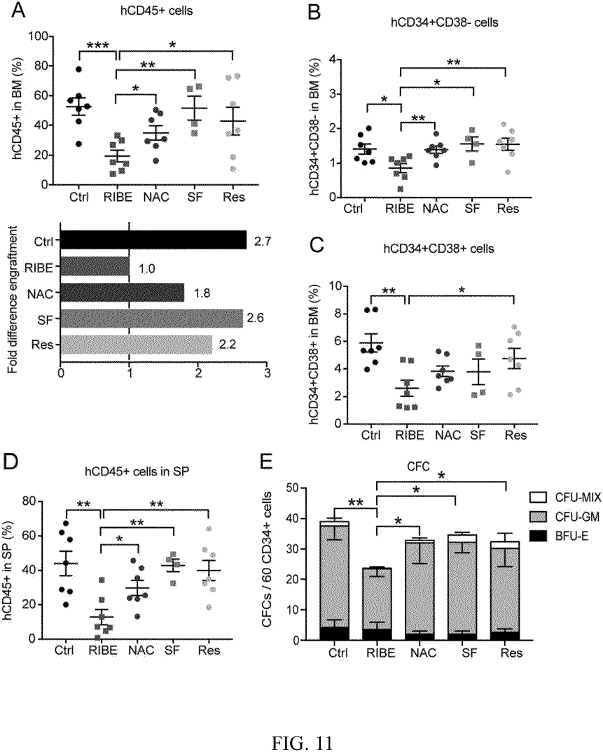

FIG. 10 is an experimental diagram of in vivo antioxidant treatment RIBE model. NOG mice were given antioxidants for 7 days before irradiation.

FIG. 11 shows that inhibition of ROS elevation protects the human HSPCs from functional deterioration in an in vivo RIBE model, wherein

All of the following procedures were consistent with those shown in FIG. 1 (n = 4 to 7 per group); and the sub-figures shown are: (A) Mean human cell engraftment (above) and bars represent the fold difference of engraftment levels between each group (below); (B-C) Frequency of human CD34⁺CD38⁻ cells (B) and CD34⁺CD38⁺ cells (C) in the BM of recipient; (D) Mean engraftment level of human cells in the spleen of recipients; and (E) Number of hematopoietic clones formed by human CD34⁺ cells of each group (^{∗}p < 0.05, ^{∗∗}p < 0.01, ^{∗∗∗}p < 0.001).

### DETAILED DESCRIPTION

### 1. Models Constructing

For the in vivo RIBE model, CD34⁺ cells purified from human cord blood (CB) were intravenously injected into 10 Gy irradiated (RIBE group) or nonirradiated (Control group) NOD/Shi-scid/IL-2Rγ^{null} (NOG) mice separately. After 17 hours, homing human CD34⁺ cells (in vivo bystander cells) from recipients were individually detected or sorted for subsequent experiments (FIG. 1).

For the in vitro RIBE model, we cocultured CB-CD34⁺ cells with 10 Gy irradiated or nonirradiated human BM cells using a transwell system. After 17 hours of coculture, the CD34⁺ cells in the insert (in vitro bystander cells) were collected for subsequent experiments (FIG. 2).

### 2. Bone Marrow Transplantation Assays

Female NOG mice were irradiated at a dose of 2.0 Gy one day before transplantation. The homing human CD34⁺ cells were transplanted into recipients at a dose of 5,000 cells/mouse intravenously. For serial transplantations, 1×10⁷ whole BM cells from each primary recipient were intravenously transplanted into secondary recipient mice that were exposed to sublethal irradiation (2.0 Gy).

### 3. In Vivo RIBE Significantly Dampen Human Long-Term Hematopoietic Reconstitution and Clonogenic Ability

The homing cells from irradiated and nonirradiated recipients were flow-sorted, and transplanted at equivalent cell doses into sublethally (2 Gy) irradiated NOG mice intravenously. No significant differences are found in the percentage of homing CD34⁺ cells between groups.

We observed that the percentage of human CD45⁺ cells in the RIBE group was significantly lower than that in the control group as early as 4 weeks after transplantation. 20 weeks after transplantation, human CD45⁺ cell engraftment of the RIBE group decreased by 2.7- and 2.3-fold in the BM and SP, respectively, compared to the control group (FIGS. 3A-B).

Importantly, the enrichment of hematopoietic stem cell (HSC, CD34⁺CD38⁻) and hematopoietic progenitor cells (HPC, CD34⁺CD38⁺) cells in the RIBE group were lower than in the control group (FIGS. 3C-D), suggesting a negative effect of irradiated recipients on donor HSCs/HPCs. The majority of human cells in the BM were CD19⁺ B cells in all the NOG mice. The control group had significantly decreased erythroid engraftment than the RIBE group (FIG. 3E).

We performed parallel secondary transplantations from both RIBE and control primary recipients. Interestingly, human cells from primary RIBE recipients generated a 33.1-fold decrease of the mean engraftment levels in the BM compared with those from control group (FIG. 3F). These data demonstrated that RIBE abrogates the long-term engraftment potential of hematopoietic stem and progenitor cell (HSPC).

The cells that initiated engraftment in xenotransplants were operationally defined as SCID-repopulating cells (SRCs). The SRC assay provided a direct quantitative in vivo assay to measure human HSC activity and engraftment. We performed LDA to measure the frequency of SRCs. 1 in 2,979 cells in the RIBE group clonally initiated long-term hematopoiesis in NOG mice, whereas 1 in 1,416 cells did so in the control group (FIG. 3G). In addition, colony-forming cell (CFC) assay showed that the number of CFCs in the RIBE group significantly decreased in comparison to that in the control group (FIG. 3H). These data revealed that the clonogenic potential of human CD34⁺ cells was suppressed after exposure to irradiated recipients.

### 4. RIBE Obstructs HSPCs′ Cell Cycle Entry and Increases Their Apoptosis And Senescence in Vivo

The hematopoietic system is the most sensitive one to irradiation, and irradiation leads to acute hematopoietic damage by inducing cell death of all the hematopoietic tissues. To explore the mechanisms underlying the impaired long-term engraftment of bystander hematopoietic cells, we firstly examined the cell cycle status of bystander human CD34⁺ cells by bromodeoxyuridine (BrdU) incorporation. The proportion of BrdU⁺ cells in the CD34⁺ cells of the RIBE group was lower than that of the control group, suggesting that cell cycle arrest occurred in bystander human HSPCs (FIG. 4A). The fractions of apoptotic cells of CD34⁺ cells in the RIBE group were increased (FIG. 4B). Furthermore, senescence-associated beta-galactosidase (SA-β-gal) staining revealed a significant increase in the senescence of CD34⁺ cells in the RIBE group compared with the control group (FIG. 4C). Altogether, the observed impaired long-term engraftment of bystander hematopoietic cells is partially attributed to the altered cell cycle status, apoptosis and senescence of HSPCs.

Next, we speculated that irradiation-induced oxidative stress may contribute to RIBE. To validate this hypothesis, we evaluated the reactive oxygen species (ROS) levels of bystander human HSPCs by analysis of oxidation of 2’,7’-dichlorofluorescin diacetate (DCF-DA) and dihydroethidium (DHE). A marked increase in ROS levels was observed in bystander human HSPCs (FIGS. 4D-E). In addition, mitochondrial ROS levels were significantly elevated in bystander human HSPCs (FIG. 4F), resulting in impaired mitochondrial membrane potential (FIGS. 4G-H).

### 5. In Vitro RIBE Causes Human HSPCs to Undergo Defects Similar to the In Vivo RIBE

The number of bystander human HSPCs that can be collected from the in vivo RIBE model is limited. To better understand the mechanisms of RIBE, we designed in vitro experiments by coculturing human CD34⁺ cells with irradiated or nonirradiated human BM cells in a transwell system. The reconstitution activity and clonogenic potential of CD34⁺ cells were evaluated in both the RIBE and control groups. There was a decreased level of human cell engraftment in the peripheral blood (PB) in the RIBE group. Human CD45⁺ cell engraftment in the BM of the RIBE group was 1.9-fold lower than that in the control group (FIG. 5A). Additionally, compared to the control group, with a lower level of HSC-enriched cells, the proportions of HPCs were slightly decreased in the RIBE group. This was due to HSCs being more sensitive to radiation and oxidation than HPCs. Similar to the in vivo model, the majority of human cells were CD19⁺ B cells in the BM of all NOG mice. However, unlike in vivo RIBE, the in vitro bystander human HSCs did not display erythroid differentiation defects, which may be caused by the absence of cell-to-cell contacts of the in vitro RIBE model. As observed in the BM, the CD45⁺ cells in the SP of RIBE group were significantly fewer than those in the control group and the majority of cells in the SP were CD19⁺ B cells. These experiments indicated that the long-term repopulating capacity of bystander human HSPCs was significantly reduced in the in vitro RIBE model.

To further assess the self-renewal potential of in vitro bystander human HSCs, we performed parallel secondary transplantation and SRC assays. Compared with that in the control group, the mean engraftment level of human cells from primary recipients in the RIBE group showed a 3.7-fold decrease (FIG. 5B). The majority of human cells were CD19⁺ B cells in the BM of all secondary NOG mice. LDA showed that 1 in 977 cells in the RIBE group clonally initiated long-term hematopoiesis compared to 1 in 638 cells in the control group. Moreover, the number of CFCs in the RIBE group significantly decreased compared to that in the control group (FIG. 5C). Taken together, these data indicated that, although not as strong as the in vivo RIBE, the long-term repopulating capacity and clonogenic potential of human HSPCs were also dampened after exposure to irradiated BM cells, further confirming the negative bystander effects of irradiated BM cells on human HSPCs.

We next detected the cell-cycle status and apoptosis status of in vitro bystander human CD34⁺ cells and demonstrated arrest cell-cycle status (FIG. 5D) and increased apoptosis in the in vitro bystander human HSPCs (FIG. 5E). Furthermore, cellular senescence detected by SA-β-gal staining showed that senescent cells accumulated in the RIBE group compared with that in control group (FIG. 5F). These results were confirmed by SA-β-gal enzymatic activity assay as shown in microscopic images. Taken together, these data indicated that RIBE caused cell-cycle arrest, apoptosis and senescence in human HSPCs.

We further examined mitochondria and energy metabolism in bystander human HSPCs. Our data showed an elevated mitochondrial ROS level of in vitro bystander human CD34⁺ cells, resulting in impaired mitochondrial membrane potential (FIGS. 5G-I). Furthermore, transmission electron microscopy (TEM) analysis showed that the mitochondria in bystander human CD34⁺ cells were swollen and round, and the number of elongated mitochondria was significantly decreased, accompanied by prevalent mitophagosome formation. Mitochondrial dysfunction leads to a reduction in ATP synthesis due to the disruption of energy metabolism. We thus investigated whether the energy phenotype of bystander human CD34⁺ cells was altered. Their energy metabolism was generally reduced (FIG. 5J), including aerobic respiration (indicated by extracellular oxygen consumption rate, OCR) (FIG. 5K) and glycolysis (indicated by extracellular acidification rate, ECAR) (FIG. 5L), which led to a reduction in intracellular ATP (FIG. 5M). These results suggest that the mitochondria in bystander human HSPCs display severe dysfunction.

### 6. Excessive ROS in Bystander Human Hematopoietic Cells Leads to DNA Damage in HSPCs

Our data in FIGS. 3 and 5 showed that the damage to human HSPCs caused by the in vitro RIBE was consistent with the in vivo RIBE. Therefore, the in vitro model was appropriate for the study of detailed mechanisms. Our further studies showed that the ROS level was also elevated in the in vitro bystander HSPCs (FIG. 6A). To elucidate the molecular mechanism of RIBE, we performed RNA-seq of the in vitro human CD34⁺ cells in both the RIBE and control groups. Gene set enrichment analysis revealed that there was activation of the p53 signaling pathway, negative regulation of the cell cycle, and positive regulation of the apoptotic signaling pathway in HSPCs in the RIBE group. Real-time qPCR results showed the upregulation of DNA damage response (DDR) markers including ATM, CHK1 and CHK2, P53; the cell-cycle inhibitors P16^{INK4a} and P21^{CIP1262}; and apoptosis-related caspases in bystander HSPCs. Further analyses by WB and immunofluorescence assays revealed cytological evidence of activated DDR in human HSPCs cocultured with irradiated BM. The activated forms of ATM, p53 binding protein, forkhead box O3a, γ-H2AX, etc. were detected by WB or immunofluorescence (FIGS. 6B-C). Furthermore, the protein expression of the cell cycle and apoptotic signaling pathway confirmed that the activation of the p53 signaling pathway by DDR led to negative regulation of the cell cycle and positive regulation of the apoptotic signaling pathway (FIGS. 6B-C). ROS levels of HSPCs were elevated as soon as 30 minutes after co-culture with irradiated bone marrow (FIG. 6D) However, the level of γ-H2AX was enhanced at about two hours after co-culture (FIG. 6E). Thus, our data suggest that the activation of a series of DDR pathways was initiated by increased ROS levels in bystander human HSPCs. Abundant evidence indicates that HSPCs are highly sensitive to ROS, which can cause oxidative DNA damage and lead to cell damage. Our data confirmed that RIBE induced excessive increases in ROS and oxidative DNA damage in bystander human HSPCs, which may lead to their exhaustion.

Cytokines may exert multiple functions involving stress and inflammatory responses, which are also associated with RIBE. We observed several cytokines including IL-1, IL-6, IL-8, TNF-α, etc., were increased after irradiation (FIG. 6F). Interestingly, the mRNA and protein levels of IL-1ra were significantly elevated after irradiation (FIG. 6G). Moreover, we added IL-1α or IL-1ra to the in-vitro culture system and then measured the γ-H2AX protein by Western blot. Based on the protein levels, we found that IL-1α could loosen DNA damage of HSPCs both in the control and RIBE groups, and IL-1ra can slightly increase DNA damage of bystander HSPCs (FIG. 6H). However, ROS levels of HSPCs did not change in either IL-1α or IL-1ra treated groups, suggesting that the alterations of DNA damage induced by these cytokines may not be attributable to ROS levels

### 7. Inhibition of ROS Elevation Rescues Human HSCs From Functional Deterioration

Next, we asked whether the pharmacological inhibition of ROS elevation could protect human HSPCs from functional degradation. We used three antioxidants: N-acetyl-L-cysteine (NAC), sulforaphane (SF) and resveratrol (Res), to eliminate excessive ROS from bystander human HSPCs in vitro. For the in vitro RIBE, the BM cells were treated with NAC, SF or Res for 30 minutes before irradiation, and the antioxidants continued to exist in the coculture system until the CB CD34⁺ cells were collected for analysis and some were transplanted into sublethally irradiated NOG mice intravenously (FIG. 7).

Oxidations of DCF-DA of groups treated with the antioxidants were significantly lower than that of untreated group, and similar with the blank group (FIG. 8A). In addition, treatment with these antioxidants prevented DNA damage (FIGS. 8B-C) and reduced apoptosis (FIG. 8D) of in vitro bystander human HSPCs. However, combination of these antioxidants did not exhibit further improvement (FIGS. 8A-D).

Then, we attempted to rescue bystander human HSPCs from functional exhaustion in long-term repopulation and clonogenic potential through treatment with antioxidant alone both in vitro and in vivo. 20 weeks after transplantation, the engraftment of bystander human CD45⁺ cells was improved by antioxidants treatment (FIG. 9A). Furthermore, we found that antioxidants could also improve HSC-enriched cell engraftment, but only SF and Res could improve HPC engraftment (FIGS. 9B-C). The majority of human cells were CD19⁺ B cells in the BM of all NOG mice, whereas the Res group had significantly higher myeloid and erythroid engraftment than the RIBE group, suggesting that Res administration in vitro had positive effects on myeloid and erythroid differentiation. As was observed in the BM, the antioxidants could also improve the in vitro bystander human cell engraftment in the SP (FIG. 9D). Secondary transplantation demonstrated that antioxidants could partially improve human HSPC long-term repopulation compared to that in the RIBE group. The LDA assay showed that HSPCs in antioxidant-treated groups contained more long-term repopulating cells than the RIBE group. Additionally, we used a CFC assay to evaluate the clonogenic potential of in vitro antioxidant-treated bystander CB-CD34⁺ cells. The number of CFCs significantly increased after antioxidant treatment (FIG. 9E).

For the in vivo RIBE, the mice were treated with NAC, SF or Res for 7 days. CB CD34⁺ cells were injected into nonirradiated or 10 Gy irradiated NOG mice in either the control, RIBE or RIBE with antioxidant treatment groups. After 17 hours, the homing human CD34⁺ cells were sorted individually and transplanted into sublethally irradiated NOG mice (FIG. 10). At 20 weeks, the transplantation results demonstrated that these antioxidants could also improve in vivo bystander human cell engraftment (FIG. 11A). The engraftment of HSC-enriched cells was improved (FIG. 11B), and unlike treatment in vitro, only Res could improve HPC engraftment in vivo (FIG. 11C). Furthermore, antioxidants used in vivo showed that the majority of human cells in the BM were CD19⁺ B cells in NOG mice, and antioxidants could not improve the erythroid potential of human HSPCs within the in vivo RIBE model. The antioxidants could also improve in vivo bystander human HSPC engraftment in the SP, as observed in the BM (FIG. 11D). Moreover, we assessed the clonogenic potential of in vivo antioxidant-treated bystander CB CD34⁺ cells and found that the number of CFCs significantly increased after antioxidant treatment (FIG. 11E). Secondary transplantation and LDA assay showed that antioxidants could partially improve human HSPC long-term repopulation activity impaired by RIBE. Taken together, our data show that the impaired long-term engraftment and clonogenic potential of bystander human HSPCs can be restored by antioxidants. These results show that the excessive ROS level is the primary cause of DNA damage in bystander human HSPCs, and the pharmacological inhibition of ROS elevation effectively prevents deterioration of human HSPC function both in vitro and in vivo.

What is claimed is:

1. An antioxidant for use in treatment for a radiation-induced bystander effect to a transplanted cell in a host.

2. The antioxidant according to claim 1, wherein the antioxidant is N-acetyl-L-cysteine, sulforaphane or resveratrol, or a combination thereof.

3. The antioxidant according to claim 1, wherein the radiation-induced bystander effect is in vivo or in vitro.

4. The antioxidant according to claim 3, wherein the transplanted cell is a hematopoietic stem cell.

5. The antioxidant according to claim 4, wherein the radiation-induced bystander effect to the transplanted cell is DNA damage or apoptosis of the transplanted cell caused by radiation to the host, or a combination thereof.

6. The antioxidant according to claim 4, wherein the radiation-induced bystander effect to the transplanted cell is the transplanted cell’s reducing engraftment in the host.

7. The antioxidant according to claim 6, wherein the radiation-induced bystander effect to the transplanted cell is a reduced hematopoietic stem cell enriched engraftment or a reduced hematopoietic progenitor cell enriched engraftment, or a combination thereof, in BM or SP, caused by radiation to the host.

8. The antioxidant according to claim 7, wherein the antioxidant is sulforaphane or resveratrol, or a combination thereof.

9. The antioxidant according to claim 3, wherein the radiation-induced bystander effect to the transplanted cell is the transplanted cell’s reduced erythroid differentiation potential.

10. The antioxidant according to claim 9, wherein the antioxidant is resveratrol.

11. The antioxidant according to claim 3, wherein the radiation-induced bystander effect to the transplanted cell is a reduced long-term repopulation potential or a reduced clonogenic potential in secondary transplantation.
